(19) United States
(12) Patent Application Publication
(10) Pub. No.: US 2025/0152566 A1
(43) Pub. Date: May 15, 2025

(54) USE OF AMINOPYRIDINE, IN PARTICULAR AMIFAMPRIDINE, FOR THE TREATMENT OF A VIRALLY-ASSOCIATED FATIGUE

(71) Applicant: Thomas BOEHMEKE, Gelsenkirchen (DE)

(72) Inventors: Thomas BOEHMEKE, Gelsenkirchen (DE); Ulrike BOEHMEKE, Gelsenkirchen (DE)

(73) Assignee: Thomas BOEHMEKE, Gelsenkirchen (DE)

(21) Appl. No.: 18/509,907

(22) Filed: Nov. 15, 2023

(51) Int. Cl. A61K 31/44; A61P 25/00

(52) U.S. Cl. CPC A61K 31/44; A61P 25/00

(57) ABSTRACT

The present invention relates to a new use of an Aminopyridine, such as Pyridine-3,4-diamine, a derivative thereof and/or of a physiologically tolerable salt thereof for the prevention and/or treatment of a fatigue, which is associated with a viral infection, preferably by SARS-CoV-2. In particular for the treatment of fatigue in connection with a “post-COVID phase” and the “long COVID-19 syndrome”.

The present invention relates to a new use of an Aminopyridine, such as, in particular Pyridine-3,4-diamine, one of its derivatives and/or a physiologically tolerable salt thereof and/or an Aminopyridine, in particular Pyridine-3,4-diamine for the use as medicament in the prophylaxis and/or treatment of a fatigue, which is associated with a viral infection, preferably from SARS-CoV-2. In particular for the treatment of fatigue in connection with a “post-COVID phase” and the “long COVID-19 syndrome” as well as preferably in combination with diseases, such as a neuroimmunological disease and/or a chronic fatigue syndrome. Pyridine-3,4-diamine, a physiologically compatible salt, solvate, hydrate, co-crystal, polymorph thereof and/or mixtures containing at least two of the compounds for the use for the prophylaxis and/or treatment of a fatigue, which is associated with a viral infection, which is triggered by ss(+) RNA viruses of the family Coronaviridae and optionally in combination for the prophylaxis and/or treatment of a fatigue, which is associated with MSC and/or LEMS and/or other infectious diseases, such as the infectious mononucleosis, is likewise an object of the invention.

It is known that in addition to somatic changes, such as heart failure, limited lung function after Corona-related pneumonia as well as complications resulting from venous or arterial thromboses, the fatigue symptomatology after COVID infection represents a particular challenge. The existing research focusses on immunomodulatory forms of therapy, such as monoclonal antibodies under the assumption of an immunologically triggered secondary complication. Clear scientific results are currently not available in spite of accelerated research.

The fatigue syndrome refers to a feeling of persistent tiredness, exhaustion and listlessness. It has a lasting adverse effect on the life of those affected and can also not be remedied by a lot of sleep. In contrast to the independent and rare disease of the chronic fatigue syndrome, the fatigue syndrome is also a side effect of chronic diseases, such as cancer, rheumatism and HIV/Aids or a result of extraordinary stresses, such as chemotherapies. The fatigue symptomatology is furthermore also known as part of general diseases, such as anemia, disorders of thyroid gland function, cardiac diseases as well as tumor diseases, but also in the case of neuromuscular diseases, such as multiple sclerosis (MS), Myasthenia gravis (MG) as well as the Lambert-Eaton myasthenic syndrome (LEMS). For many patients with these diseases, the fatigue symptomatology are the main focus, the limited mobility as a result of muscular deficits is often perceived to be secondary. A specific therapy with Aminopyridine exists for LEMS and displays no impairments of the liver and kidney function in the everyday clinical practice up to a daily maximum dose of 60 mg.

Fatigue is thus a pathological, disproportionate exhaustion, which cannot be eliminated with sleep, which can be of a physical as well as mental and/or emotional nature. This exhaustion does not originate from a previous exertion. It can thus be clearly separated from the physiological tiredness. The fatigue leads to a decreased cerebral blood circulation and ineffective oxygen utilization and thus to the failing performance and ability to recover. An adrenergic hyperstimulation with tachycardia, hypervigilance and ineffective sensory stimuli occurs simultaneously.

The degree of severity can be captured by means of questionnaires about the general functional impairment (Bell score), about the quality of life (e.g., EQ5D, PedsQL, SF-36) and about individual symptoms (fatigue severity scale [FSS] as well as Chalder fatigue scale).

As part of the COVID-19 pandemic, it was now found that a portion of the cases of long COVID and post-COVID-19 syndrome—also in childhood and adolescence—clinically progresses or is diagnosed, respectively, as myalgic encephalomyelitis/chronic fatigue syndrome (ME/CFS).

The fatigue symptomatology can currently only be captured clinically, clear and reliable laboratory parameters are currently not described. Fatigue manifests itself in an increased need for sleep and rest, whereby a lack of recovery even after several hours of sleep during the day with increased duration of nightly sleep is typical. There is furthermore often a significantly limited endurance, climbing one flight of stairs is typically only possible with difficulty, without being able to confirm a correlating cardiac, pulmonary or other internal problem. A manifest fatigue represents a serious limitation not only of coping with daily tasks but also for pursuing one's profession.

It is the object of the invention to provide a remedy, which is suitable to treat and to reduce the symptoms caused by a COVID infection, such as the fatigue symptom. It is in particular the object of the present invention to provide a new therapy for treating patients, in particular of children and adults, who suffer from long COVID or post-COVID-19 syndrome. A possible prophylaxis, prevention and/or treatment is to be provided, which prevents or reduces the fatigue, improves or completely cancels the Bell score and preferably achieves a score of up to 100. An effectively acting dose or effective dose (ED) is to likewise be found for the remedy. An effectively acting dose is the dosage of the free amine, which can optionally be present as salt, co-crystal, etc.

Surprisingly, it was found that a Diaminopyridine, preferably 3,4-Diaminopyridine, can be used for the prophylaxis and/or treatment of a fatigue, which is associated with, in particular triggered by, a viral infection, and/or optionally for the prophylaxis and/or treatment of a complex neuroimmunological disease comprising myalgic encephalomyelitis and/or optionally chronic fatigue syndrome (ME/CFS), preferably a post-infectious fatigue syndrome. Particularly preferably with an effective dose with regard to the active ingredient Diaminopyridine, in particular 3,4-Diaminopyridine, of one to three doses of 5 mg per day. A preferred total dose can be 5 mg to 15 mg per day. Depending on the patient, a single dose of 5 mg per day is taken before going to sleep, in particular in the evening. In the evening is considered to be a time from about 6 pm, and before going to sleep is considered to be about 0 to 2 hours before going to sleep, in particular 0 to 60 minutes before going to sleep, particularly preferably 0 to 45 min, 30 min, particularly preferably understood to be 15 minutes before going to sleep.

Diaminopyridine, in particular Pyridine-3,4-diamine (IUPAC) or also 3,4-Diaminopyridine, 3,4-DAP (CAS-Nr: 54-96-6, Amifampridine) of the formulas:

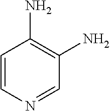

and its derivatives are known and concrete compounds are approved for treatment. By means of reversible blocking, Pyridine-3,4-diamine prevents that potassium ions leave the nerve cells. The depolarization at the presynaptic end of the nerve cells is prolonged thereby. The prolonged depolarization, in turn, has an influence on the release of acetylcholine into the synapses. The increased release of acetylcholine improves the muscle contraction. Derivatives comprise Monoaminopyridine and/or Diaminopyridine, in particular 3,4-Diaminopyridine or Pyridine-3,4-diamine, respectively, and/or 4-Aminopyridine. Amifampridine can block the voltage-gated potassium channels and can thus prolong the depolarization of the presynaptic cell membrane and can improve the release of Acetylcholine at the synapses, can improve the saltatory conduction and ultimately improve the muscle function.

Aminopyridine in terms of the invention comprises Pyridine-3,4-diamine, its derivatives, 4-Aminopyridins, Diaminopyridine, as well as the physiologically tolerable salts, Pyridine-3,4-diamine and/or as well as 4-Aminopyridine and/or 3-Aminopyridrine is preferred.

EP 0 159 112 discloses Amifampridine and the monohydrochloride salt. US 2004/0106651 discloses the production of phosphate and tartrate salt of Amifampridine and the use thereof for treating botulism, myasthenia, myasthenic syndromes and tiredness in connection with a neurological disease, e.g. multiple sclerosis or amyotrophic lateral sclerosis (ALS). EP3696169A1 discloses the production of a phosphate salt of Amifampridine.

In a first aspect, the present invention relates to a use of Pyridine-3,4-diamine, one of its derivatives and/or a physiologically tolerable salt, solvate, hydrate, co-crystal, polymorphs thereof and/or mixtures containing two of the mentioned compounds for the prophylaxis, prevention and/or treatment of a fatigue, which is associated with a viral infection.

The fatigue in terms of the invention is preferably a fatigue as described here. It is in particular a fatigue, which could analogously be diagnosed as chronic fatigue syndrome, in accordance with ICD code G93.3. A fatigue, which is associated with a viral infection, is similar or comparable with the diagnostic relating to the ICD code G93.3. However, the cause is rooted in the viral infection.

In a second aspect of the use in terms of the invention, the viral infection is triggered by ss(+) RNA viruses of the family Coronaviridae, preferably by the virus SARS-CoV-1, SARS-CoV-2, MERS-COV, HCoV-HKU1, HCoV-OC43, HCoV-NL63 and/or HCoV-229E, or by a virus of the family of the Herpesviridae comprising Epstein-Barr virus.

In a preferred aspect of the use in terms of the invention, the viral infection is a SARS-CoV-2 infection or an Epstein-Barr virus infection.

In a fourth aspect of the use in terms of the present invention, the fatigue is a post-infectious fatigue, which preferably appears after the viral infection has abated, preferably after a Ct value of greater than or equal to 15 was determined. Preferably greater than or equal to 20, greater than or equal to 25, greater than or equal to 30, greater than or equal to 35 or more. The use in terms of the invention for preventing, for avoiding and/or treating a complex neuroimmunological disease comprising myalgic encephalomyelitis and/or chronic fatigue syndrome (ME/CFS), preferably of a post-infectious fatigue syndrome, is likewise an object of the invention.

The Ct value (cycle-threshold value) specifies the number of measuring cycles, which are performed within a PRC sample in order to be able to detect the Coronavirus. The greater the Ct value, the lower the viral load in the measured sample. With increasing Ct value, the progression of the disease of the patient moves away from the acute disease phase, and the patients transitions into a post-post-infectious disease phase. The Ct value alone is not always reliable and it can be advantageous to consult the conversion of Ct/Cq values into viral RNA loads (RNA copies per sample volume) by means of calibration with the help of a standardized virus RNA preparation.

In a further aspect of the use in terms of the invention, the health problems of the acute disease phase of a viral, preferably SARS-CoV-2, infection, persist for greater than or equal to 3, greater than or equal to 4 weeks or they appear again. The patient is particularly preferably in a so-called “Post-COVID phase”, as defined in the guideline recommendations of the British National Institute for Health and Care Excellence (NICE). According to this, problems, which are still present more than 12 weeks after onset of the SARS-CoV-2 infection and which cannot be explained otherwise, are referred to as post-COVID syndrome. “Long COVID” comprises symptoms, which, following an acute COVID-19 disease, still exist 4 to 12 weeks after symptom onset as well as the “post-COVID-19 syndrome”.

It is thus a further aspect of the use according to the invention that the symptoms of the acute disease phase of a viral, preferably SARS-CoV-2, infection, continue for more than 4, preferably more than 5, more than 6, more than 7, more than 8, more than 9, more than 10, more than 11 or more than 12 weeks after onset of the viral, preferably SARS-CoV-2, infection. The patient in particular has a “long COVID-19 syndrome”, in particular according to the guideline recommendations of the British National Institute for Health and Care Excellence (NICE). The NICE guidelines [NG188] were published on Dec. 18, 2020 and were last updated on Nov. 11, 2021. “More than” is to be understood synonymously with “greater than or equal to”.

An acute disease phase of a viral infection in terms of the present invention comprises symptoms, such as cough, fever, cold, impaired sense of smell and/or taste, sore throat, shortness of breath, headache and body aches, loss of appetite, weight loss, nausea, abdominal pain, vomiting, diarrhea, conjunctivitis, skin rash, swollen lymph nodes, apathy and/or somnolence. Not all of the symptoms have to appear simultaneously and not all together. Each patient has an individual symptomatology, which can have one or several of the above-mentioned symptoms simultaneously or one after the other.

In a further aspect of the use according to the invention, the fatigue comprises symptoms selected from sleep disorders, tiredness, increased need for sleep, muscular weakness, decreased physical endurance, decreased mental endurance, decreased recollection ability, impairment in the ability to think and remember, loss of concentration, increased pain perception and/or an attention deficit and/or dysregulations of the circulation, hormone and/or of the immune system. The symptoms can be limited to symptoms determined by means of a survey, but can also appear in combination with biochemical parameters, which are used to analyze circulation, hormone and/or immune system. These parameters are known to the person of skill in the art and comprise the measurement of the hormones, e.g., melatonin, cortisol and/or serotonin, the measurement of the upper, systolic and the lower, diastolic value, determination of the blood sugar, etc.

In a further aspect of the use according to the invention, a fatigue is present with a Bell score of less than or equal to 50, less than or equal to 40, less than or equal to 30, less than or equal to 20. To determine the Bell score, the respective current state of the patient is determined relative to the state of the patient at a point in time prior to the viral infection.

In a further aspect of the use according to the invention, in particular by means of the administration of the at least one effective Pyridine-3,4-diamine, derivatives and/or at least one physiologically tolerable salts, the need for sleep is reduced to physiological hours of greater than or equal to 6 to less than or equal to 12, preferably greater than or equal to 6, greater than or equal to 7, greater than or equal to 8 to less than or equal to 11 hours, less than or equal to 10, less than or equal to 9, particularly preferably greater than or equal to 6 to less than 8.

A further aspect of the present invention is a composition containing at least one Pyridine-3,4-diamine, at least one of its derivatives and/or at least one physiologically tolerable salt for the use in the prevention, prophylaxis, protection, avoidance of the formation and/or treatment of a fatigue as described herein, which is associated with a viral infection. A method for treating, preventing the formation and/or avoiding the development of a complex neuroimmunological disease comprising myalgic encephalomyelitis and/or chronic fatigue syndrome (ME/CFS), preferably of a post-infectious fatigue syndrome, is likewise a subject matter of the invention.

Pyridine-3,4-diamine, a physiologically tolerable salt, solvate, hydrate, co-crystal, polymorph thereof and/or mixtures containing at least two of the compounds for the use for the prophylaxis and/or treatment of a fatigue, which is associated with a viral infection, which is triggered by ss(+) RNA viruses of the family Coronaviridae, and optionally in combination for the prophylaxis and/or treatment of a fatigue, which is associated with MSC and/or LEMS and/or other infectious diseases, such as the infectious mononucleosis and/or influenza viruses, in particular influenza viruses from the family of the Orthomyxoviridae, which can trigger the disease influenza, is likewise a subject matter of the invention. The influenza viruses are enveloped viruses with single-stranded, segmented RNA of negative polarity as genome (+/− ssRNA).

The following genus are assigned to the influenza viruses: alpha, beta, gamma and delta influenza virus. The species preferably comprise influenza A virus (FLUAV) to influenza D virus (FLUDV).

3,4-Diaminopyridine or Pyridine-3,4-diamine, respectively, a physiologically tolerable salt, solvate, hydrate, co-crystal, polymorphs thereof and/or mixtures containing at least two of the mentioned compounds for the use for the prophylaxis and/or treatment of a fatigue, as described herein, which is associated with a viral infection and/or for the prophylaxis and/or treatment of a complex neuroimmunological disease comprising myalgic encephalomyelitis and/or chronic fatigue syndrome (ME/CFS), preferably of a post-infectious fatigue syndrome, is a subject matter of the invention. The use as described herein is preferred. Particularly preferably of greater than or equal to 2 mg in 24 hours with regard to the active ingredient 3,4-Daminopyridine, more preferably of greater than or equal to 5 mg in 24 hours up to 100 mg within 24 hours with regard to the active ingredient 3,4-Diaminopyridine. According to a particularly preferred alternative of an administration of at least twice a day, in particular within 24 hours, of greater than or equal to 2 mg each, preferably of 5 mg each with regard to the active ingredient. A particularly preferred daily dose within 24 hours is 0 to 15 mg, in particular 5 mg to 15 mg, preferably 5 mg of active ingredient each per single dose. It is particularly preferred thereby to use one to three single doses of 1 to 10 mg active ingredient each, preferably of 2 mg to 7 mg, particularly preferably of 5 mg plus/minus 2 mg, preferably of plus minus 1 mg per single dose, as daily dosage, in particular to use them for the disclosed purposes.

A method for the prophylaxis and/or for the treatment of a fatigue, which is associated with a viral infection and/or a complex neuroimmunological disease comprising myalgic encephalomyelitis and/or chronic fatigue syndrome (ME/CFS), preferably of a post-infectious fatigue syndrome with 3,4-Diaminopyridine or Pyridine-3,4-diamine, respectively, a physiologically tolerable salt, solvate, hydrate, co-crystal, polymorphs thereof and/or mixtures containing two of the mentioned compounds, is a subject matter of the invention.

Post-infectious fatigue syndrome is understood to be a fatigue syndrome, which is associated with a viral infection, in particular comprising viral infections, with one or several of the viruses SARS-CoV-1, SARS-CoV-2, MERS-COV, HCoV-HKU1, HCoV-OC43, HCoV-NL63 and/or HCoV-229E, or of the family of the Herpesviridae comprising Epstein-Barr virus, which was in particular triggered and/or intensified by a viral infection.

Pharmaceutical composition comprising, in particular a potassium channel blocker, preferably Pyridine-3,4-diamine, in particular as active ingredient, a physiologically tolerable salt, solvate, hydrate, co-crystal, polymorphs thereof and/or mixtures containing two of the mentioned compounds as well as at least one pharmaceutical excipient for the use for the prophylaxis and/or treatment of a fatigue as described herein, which is associated with a viral infection and/or for the prophylaxis and/or treatment of a complex neuroimmunological disease comprising myalgic encephalomyelitis and/or chronic fatigue syndrome (ME/CFS), preferably of a post-infectious fatigue syndrome. A pharmaceutical compound for use according to any one of claims 1 to 13 is particularly preferred. Pharmaceutical composition comprising greater than or equal to 2 mg to 15 mg with regard to the active ingredient Pyridine-3,4-diamine, preferably of 5 mg with plus/minus 5% by weight with regard to the active ingredient Pyridine-3,4-diamine. Pharmaceutical composition comprising 1 mg, greater than or equal to 2 mg, greater than or equal to 3, preferably greater than or equal to 5 mg to less than or equal to 60 mg of Pyridine-3,4-diamine. The specification in mg thereby refers to the free base, to the base in the salt, the base in the co-crystal, etc.

3,4-Diaminopyridine or Pyridine-3,4-diamine, respectively, a physiologically tolerable salt, solvate, hydrate, co-crystal, polymorphs thereof and/or mixtures containing two of the mentioned compounds for the use for the prophylaxis and/or treatment of a fatigue in connection with a “post-COVID phase” and the “long COVID-19 syndrome” as well as preferably in combination with diseases, such as a neuroimmunological disease and/or of a chronic fatigue syndrome, is a subject matter of the invention. Pyridine-3,4-diamine, a physiologically tolerable salt, solvate, hydrate, co-crystal, polymorph thereof and/or mixtures containing at least two of the compounds for the use for the prophylaxis and/or treatment of a fatigue, which is associated with a viral infection, which is triggered by ss(+) RNA viruses of the family Coronaviridae and optionally in combination for the prophylaxis and/or treatment of a fatigue, which is associated with MSC and/or LEMS and/or other infectious diseases, such as infectious mononucleosis, is likewise a subject matter of the invention.

Physiologically tolerable salts are understood to be salts of the phosphoric acid, with phosphate(s), phosphate ester, HX, wherein HX comprises X⁻ comprising chloride, bromide, iodide and with H in HX equal to H⁺, zinc salts, as well as hydrates and/or solvates of these salts of Pyridine-3,4-diamine. Dihydrohalogenides, such as dihydrochloride as well as the hydrates, solvates and/or mixtures thereof are preferred. Phosphate esters comprise esters of the orthophosphoric acid (H₃PO₄). Zinc, alkaline earth and/or alkali metal salts of polycarboxylic acids can likewise be used as physiologically tolerable salts.

Alcohol or polyalkylene, with alkylene with 1 to 4 C atoms containing molecular complexes of Pyridine-3,4-diamine as well as of the salts of Pyridin-3,4-diamine are considered to be solvates of Pyridine-3,4-diamine. Preferred alcohols comprise ethanol, isopropanol and/or butanol.

Stoichiometric and non-stoichiometric amorphous or crystalline molecular compounds comprising Pyridine-3,4-diamines and water are considered to be hydrates.

Salts with organic carboxylic acids, in particular hydroxy-functional carboxylic acids, fruit acids, fatty acids, mono- or dicarboxylic acids, tricarboxylic acids, unsaturated mono-, di- and/or tricarboxylic acids or salts with alkali ions and/or alkaline earth ions with at least one carboxylate of the above-mentioned carboxylic acids are understood to be physiologically tolerable salts. Suitable carboxylic acids comprise saturated or unsaturated mono-, di- or tri-carboxylic acids with 1 to 15 C atoms, such as lactic acid, fumaric acid, succinic acid, glutaric acid, adipic acid, citric acid and/or the carboxylates thereof, salts with alkali and/or alkaline earth ions and mixtures comprising at least two of the mentioned compounds.

Co-crystals can comprise vitamins, such as nicotinamide, folic acid and/or ascorbic acid. Further vitamins, with which co-crystals can be produced, comprise thiamine, riboflavin, niacin, nicotinic acid amide, nicotinic acid, pantothenic acid vitamin B6, biotin, folic acid, vitamin B12, cobalamin, cholecalciferol, tocopherol, phylloquinone and/or menaquinone or mixtures thereof comprising at least two of the mentioned compounds. Further compounds, with which co-crystals can be produced, comprise: saccharin, nicotinamide, triester acid, nicotinic acid amide, 18-Crown-6, succinic acid, fumaric acid, tartaric acid, malic acid, malonic acid, benzamide, mandelic acid, glycolic acid, urea, nicotinic acid, 2,6-Pyridine carboxylic acid, 5-Nitroisophalic acid, 1,3,5,7-Adamantane-tetracarboxylic acid and/or mixtures of at least two of the mentioned compounds, optionally in combination with an HX salt.

The composition can comprise further pharmaceutical approved excipients: fillers, disintegrating agents, glide agents, explosives, lubricants, mold release agents, flow regulators, solvents, emulsifiers, solubilizers, wetting agents, salt formers, buffers, gelling agents, thickening agents, film-forming agents, binding agents, sorption agents, sweetening agents, coloring agents, softening agents, stabilizers, matrix-forming agents, polymers, acidifying agents, preservatives, fragrances and/or retarding agents. Further excipients comprise carrier substances, preservatives, antioxidants, stabilizers, vitamins, coloring agents, odor improvers and flavorings.

Flavorings serve to mask a potentially unpleasant flavor. For example, mint, anis, fennel, menthol, cumin, medicinal honey, honey, agave juice, sugar, sugar replacement as well as substitutes, propolis and other flavorings and aromatic substances known according to the prior art.

A composition containing at least one Pyridine-3,4-diamine, at least one of its derivatives and/or at least one physiologically tolerable salt, is a further subject matter the present invention, wherein the composition is present in liquid, pasty or solid form.

Liquid forms comprise solutions, dispersions, suspensions and emulsions containing at least one Aminopyridine in terms of the invention, can comprise the usual excipients, such as solvents, solubilizing agents and emulsifiers, e.g., water, ethanol, isopropanol, ethyl carbonate, ethyl acetate, benzyl alcohol, benzyl benzoate, propylene glycol, 1,3-butylglycol, oils, cottonseed oil, peanut oil, corn oil, olive oil, castor oil and sesame oil, glycerin fatty acid esters, polyethylene glycols and fatty acid esters of the sorbitan or mixtures of at least two of the above-mentioned substances. Preferred excipients for solutions, dispersions and suspensions containing the at least one Pyridine-3,4-diamine, at least one of its derivatives and/or at least one physiologically tolerable salt comprise dextrose, mannitol, tragacanth, pectin, methylcellulose, hydroxypropylmethylcellulose (HPMC), carboxymethylcellulose sodium, polyvinylpyrrolidone, polyvinyl alcohol, polyacrylic acid, polyethylene glycol, polyethylene oxide, sodium dodecyl sulfate, sodium cetyl stearyl sulfate, and/or Na-dioctyl sulfosuccinate (also K, Ca salts) and in particular also cellulose for suspensions.

In terms of the invention, the composition is present in a form, which is suitable for the oral administration comprising i) solid forms, such as tablet, powder, granulate, effervescent tablet, dry syrup, dragee, globules, capsule and lyophilizate and ii) liquid forms, such as suspension, solution, dispersion, tincture, concentrate, tea. Tablets in terms of the invention comprise simple tablets, lozenges, sublingual and buccal tablets, orally dispersible tablets, dissolving tablet, micro, chewable and effervescent tablets, point-, skeleton- and multi-layer tablets, dragees and pellets. Retarding properties have, e.g., the excipients ethyl- and methylcellulose, hydroxy propyl methyl cellulose, cellulose acetate phthalate and hydroxy propyl methyl cellulose phthalate.

In terms of the invention, a tablet is preferred, containing greater than or equal to 5 mg, greater than or equal to 10 mg of an Aminopyridine comprising Pyridine-3,4-diamine, its derivatives, 4-Aminopyridine, Diaminopyridine, compounds without NH₂ at the C4 but with NH₂ at the C3, in particular 3-Aminopyridine, as well as the tolerable salts, Pyridine-3,4-diamine and/or 3-Aminopyridine is preferred. A Diaminopyridine is particularly preferred. The respective tablet can preferably be portioned.

The dose, in particular an initial dose, is preferably 3 to 5 mg, preferably up to three times a day (i.e., 9 to 15 mg per day). The dose can increase slowly and several times a day, preferably up to four times a day (i.e., to up to 20 mg per day). The daily total dose can further be increased, 4 to 5 days 5 mg per day can be added, whereby a maximum dose should be 60 mg per day. Per day preferably means within a period of 24 h.

The compound has a shortened half-life period. It is thus recommended to administer several comparatively lower doses spread throughout the day or to use complex retarded formulations. Possible formulations in terms of the invention are listed in Table 1 below. The number 1 in Table 1 means thereby that the respective excipients in the formulation can fulfill the mentioned application or technical function.

TABLE 1

| | Application | | | | | | | | | | | |
|---|---|---|---|---|---|---|---|---|---|---|---|---|
| | | | Tablets | | | | | | | Technical function | | |
| | | | & | | | | | | | | Disinte- | |
| | Homeo- | | pellets | | | | | Semi- | | | grating | Glide |
| | pathic | | with | Hard | Soft | | | solid | | | agent, | agents, |
| Excipient | tritu- | | and | gelatin | gelatin | Solu- | Suspen- | prepa- | Pow- | Fill- | explo- | lubri- |
| Name | ration | Tea | without | capsules | capsules | tion | sion | ration | der | er | sives | cants |
| 1,2-propylene glycol | | | 1 | 1 | 1 | | | | | | | |
| Acetyl tributyl citrate | | | 1 | 1 | 1 | | | | | | | |
| Agar-agar | | | | | | 1 | | | | | | |
| Alkyl-4-hydroxybenzoate | | | 1 | 1 | 1 | 1 | 1 | | | | | |
| Aluminum-fatty acid | | | 1 | 1 | 1 | | | | 1 | | | |
| compounds | | | | | | | | | | | | |
| Aluminum oxide/ | | | | 1 | 1 | 1 | | | | 1 | | |
| hydroxide | | | | | | | | | | | | |
| Gum Arabic | | | | | | 1 | | | 1 | | | |
| Essential oils | 1 | 1 | 1 | 1 | 1 | 1 | 1 | 1 | 1 | | | |
| Bentonite | | | | | | | 1 | 1 | | | | |
| Calcium carbonate | | | 1 | 1 | | | | | | 1 | | |
| Calcium hydrogen | 1 | | 1 | 1 | | | | | | | | |
| phosphate | | | | | | | | | | | | |
| Carboxymethyl cellulose | | | 1 | 1 | 1 | 1 | 1 | 1 | | | 1 | |
| sodium | | | | | | | | | | | | |
| Carotene | | | | 1 | 1 | 1 | | | | | | |
| Cellulose | | | 1 | | 1 | | 1 | | | 1 | | |
| Cellulose acetate | | | 1 | | 1 | | | | | | | |
| phthalate | | | | | | | | | | | | |
| Cetyl alcohol | | | | | | | | 1 | | | | |
| Cetyl stearyl alcohol | | | | | | | | 1 | | | | |
| Citric acid | | | 1 | | | 1 | 1 | | | | | |
| Dextrose | | | 1 | | | 1 | 1 | | | 1 | | |
| Dibutyl/diethyl-phthalate | | | | 1 | 1 | 1 | | | | | | |
| Dimethylpolysiloxanes | | | | | | | 1 | 1 | | | | 1 |
| Iron oxides | | | 1 | 1 | 1 | | | | | | | |
| Ethyl alcohol | 1 | | 1 | 1 | 1 | 1 | 1 | | | | | |
| Ethyl cellulose | | | 1 | | 1 | | | | | | | |
| Gelatine | | | | 1 | 1 | | | | | | | |
| Glycerol triacetate | | | 1 | 1 | 1 | | | | | | | |
| Glycine | | | | | | 1 | | | | 1 | | |
| Glyerol | 1 | | 1 | 1 | 1 | 1 | 1 | | | | | |
| Glyerol monostearate | | | 1 | | | | | 1 | | | | |
| Colloidal silicon dioxide | 1 | | 1 | 1 | | | 1 | 1 | 1 | | | 1 |
| Hydroxypropyl methyl | | | 1 | 1 | 1 | 1 | 1 | 1 | | | | |
| cellulose (HPMC) | | | | | | | | | | | | |
| Hydroxypropyl | | | 1 | | 1 | | | | | | | |
| methylcellulose phthalate | | | | | | | | | | | | |
| Isopropyl alcohol | 1 | | 1 | 1 | 1 | 1 | 1 | | | | | |
| Isopropyl myristate | | | | | | | | 1 | | | | |
| Lactose | 1 | | 1 | | | | | | | 1 | | |
| Lecithin | | | | | | 1 | | 1 | | | | |
| Macrogol-1000-glycerol- | | | | | | | | 1 | | | | |
| monolaurat, | | | | | | | | | | | | |
| monostearate, | | | | | | | | | | | | |
| monooleate | | | | | | | | | | | | |
| Macrogol-1500- | | | | | | | | 1 | | | | |
| glyceroltriricinoleate | | | | | | | | | | | | |
| Macrogol gylerol hydroxy | | | | | | | | 1 | | | | |
| stearate | | | | | | | | | | | | |
| Macrogol stearate 400 | | | | | | | | 1 | | | | |
| Magnesium stearate | | | 1 | 1 | | | | | | | | 1 |
| Maltodextrin | 1 | 1 | | | | 1 | | | | 1 | | |
| Mannitol | | | 1 | | | 1 | 1 | | | 1 | | |
| Methylcellulose | | | 1 | | | 1 | 1 | | | | | |
| Sodium cetylstearyl | | | 1 | | 1 | 1 | 1 | 1 | | | | |
| sulfate | | | | | | | | | | | | |
| Sodium dioctyl | | | | | | 1 | 1 | | | | | |
| sulfosuccinate | | | | | | | | | | | | |
| (also K, Ca salts) | | | | | | | | | | | | |
| Sodium dodecyl sulfate | | | 1 | | 1 | 1 | 1 | 1 | | | | |
| Sodium hydrogen | | | 1 | 1 | | 1 | | | | | 1 | |
| carbonate | | | | | | | | | | | | |
| Oleic acid oleyl ester | | | | | | | | 1 | | | | |
| Pectin | | | | | | | 1 | 1 | 1 | | | |
| Poloxamer | | | 1 | | | 1 | 1 | 1 | | | | |
| Polyacrylic acid | | | | | | 1 | 1 | | | | | |
| Polyethylene glycol | | | 1 | 1 | 1 | 1 | 1 | 1 | | | | 1 |
| Polyethylene oxide | | | 1 | 1 | 1 | 1 | 1 | 1 | | | | 1 |
| Polymethacrylates | | | 1 | 1 | 1 | | | | | | | |
| Polyoxyl 23 lauryl ether, | | | | | | | | 1 | | | | |
| 20 cetostearyl ether, 10 | | | | | | | | | | | | |
| oleyl ether | | | | | | | | | | | | |
| Polyoxyl 40 stearate, 50 | | | | | | | | 1 | | | | |
| stearate | | | | | | | | | | | | |
| Polysorbate 20, 60, 80, 40 | | | | | | | 1 | 1 | | | | |
| Polyvinyl acetate | | | 1 | 1 | 1 | | | | | | | |
| Copolymers | | | | | | | | | | | | |
| Polyvinyl alcohol | | | 1 | 1 | 1 | 1 | 1 | | | | | |
| Polyvinyl pyrrolidone | | | 1 | 1 | 1 | 1 | 1 | | | | 1 | |
| Riboflavin | | | 1 | 1 | 1 | 1 | 1 | | | | | |
| Castor oil | | | 1 | 1 | 1 | | | | | | | |
| Saccharose | | | 1 | | | | | | | 1 | | |
| Sorbitan monooleate, | | | | | | | | 1 | | | | |
| palmitate, stearate, | | | | | | | | | | | | |
| trioleate, tristearate, | | | | | | | | | | | | |
| laurate | | | | | | | | | | | | |
| Sorbitol | | | 1 | | 1 | | | | | | | |
| Starch | | | 1 | | | | | | 1 | 1 | 1 | |
| Starches, rice, corn, | | | 1 | 1 | | | | | 1 | 1 | | |
| potato, wheat | | | | | | | | | | | | |
| Stearic acid | | | 1 | 1 | | | | 1 | | | | 1 |
| Stearyl alcohol | | | | | | | | 1 | | | | |
| Talcum | | | 1 | 1 | | | | | 1 | | | 1 |
| Titanium dioxide | | | 1 | 1 | | | | 1 | 1 | | | |
| Tragcanth | | | 1 | | | 1 | 1 | | | | | |
| Triacetin | | | 1 | 1 | 1 | | | | | | | |
| Triethanolamine | | | | | | 1 | | | | | | |
| Triethyl citrate | | | 1 | 1 | 1 | | | | | | | |
| Trometamol | | | | | | 1 | | | | | | |
| Vaseline | | | | | | | | 1 | | | | |
| Tartaric acid | | | 1 | | | 1 | 1 | | | | | |
| Urea | | | | | | | | | | | | |
| Ceramide | | | | | | | | | | | | |
| Hyaluronic acid | | | | | | | | | | | | |

| | Technical function | | | | | | | | | |
|---|---|---|---|---|---|---|---|---|---|---|
| | | | | Gelling | | | | | | |
| | | | | agents, | | | | | Basis, | |
| | Solvents, | Emul- | | thick- | | | | | e.g., for | |
| | solution | sifier, | Salt | ening | Sorp- | Sweet- | Color- | Soft- | powder, | |
| Excipient | acceler- | solubi- | formers, | agents, | tion | ening | ing | ening | matrix- | Stabi- |
| Name | ators | lizers | buffers | film- | agents | agents | agents | agents | forming | lizer |
| 1,2-propylene glycol | | | | | | | | 1 | | |
| Acetyl tributyl citrate | | | | | | | | 1 | | |
| Agar-agar | | | | 1 | | | | | | |
| Alkyl-4-hydroxybenzoate | | | | | | | | | | 1 |
| Aluminum-fatty acid | | | | | | 1 | | | | |
| compounds | | | | | | | | | | |
| Aluminum oxide/ | | | | | | | | | | 1 |
| hydroxide | | | | | | | | | | |
| Gum Arabic | | | | 1 | | | | | | |
| Essential oils | | | | | | | | 1 | | |
| Bentonite | | | | 1 | | | | | | |
| Calcium carbonate | | | 1 | | | | | | | |
| Calcium hydrogen | | | | 1 | | | | | | |
| phosphate | | | | | | | | | | |
| Carboxymethyl cellulose | | | | 1 | | | | | | |
| sodium | | | | | | | | | | |
| Carotene | | | | | | | 1 | | | |
| Cellulose | | | | | 1 | | | | | 1 |
| Cellulose acetate | | | | 1 | | | | | | |
| phthalate | | | | | | | | | | |
| Cetyl alcohol | | 1 | | | | | | | | |
| Cetyl stearyl alcohol | | 1 | | | | | | | | |
| Citric acid | | | 1 | | | | | | | |
| Dextrose | | | | | | 1 | | | | |
| Dibutyl/diethyl-phthalate | | | | | | | | 1 | | |
| Dimethylpolysiloxanes | | | | | | | | | | |
| Iron oxides | | | | | | | 1 | | | |
| Ethyl alcohol | 1 | | | | | | | | | |
| Ethyl cellulose | | | | 1 | | | | | 1 | |
| Gelatine | | | | 1 | | | | | 1 | |
| Glycerol triacetate | | | | | | | | 1 | | |
| Glycine | | | 1 | | | | | | | |
| Glyerol | 1 | | | | 1 | | | 1 | | |
| Glyerol monostearate | | 1 | | | | | | | | |
| Colloidal silicon dioxide | | | | 1 | 1 | | | | | 1 |
| Hydroxypropyl methyl | | | | 1 | | | | | 1 | |
| cellulose (HPMC) | | | | | | | | | | |
| Hydroxypropyl | | | | 1 | | | | | 1 | |
| methylcellulose phthalate | | | | | | | | | | |
| Isopropyl alcohol | 1 | | | | | | | | | |
| Isopropyl myristate | 1 | | | | | | | | | |
| Lactose | | | | | | | | | | |
| Lecithin | | 1 | | | | | | | | |
| Macrogol-1000-glycerol- | | 1 | | | | | | | | |
| monolaurat, | | | | | | | | | | |
| monostearate, | | | | | | | | | | |
| monooleate | | | | | | | | | | |
| Macrogol-1500- | | 1 | | | | | | | | |
| glyceroltriricinoleate | | | | | | | | | | |
| Macrogol gylerol hydroxy | | 1 | | | | | | | | |
| stearate | | | | | | | | | | |
| Macrogol stearate 400 | | 1 | | | | | | | | |
| Magnesium stearate | | | | | | | | | | |
| Maltodextrin | | | | | | | | | | 1 |
| Mannitol | | | | | 1 | 1 | | | | |
| Methylcellulose | | | | 1 | | | | | | |
| Sodium cetylstearyl | | 1 | | | | | | | | |
| sulfate | | | | | | | | | | |
| Sodium dioctyl | | 1 | | | | | | | | |
| sulfosuccinate | | | | | | | | | | |
| (also K, Ca salts) | | | | | | | | | | |
| Sodium dodecyl sulfate | | 1 | | | | | | | | |
| Sodium hydrogen | | | 1 | | | | | | | |
| carbonate | | | | | | | | | | |
| Oleic acid oleyl ester | 1 | | | | | | | | | |
| Pectin | | | | 1 | | | | | | |
| Poloxamer | | 1 | | | | | | | | |
| Polyacrylic acid | | | | 1 | | | | | | 1 |
| Polyethylene glycol | 1 | | | 1 | | | | 1 | 1 | |
| Polyethylene oxide | 1 | | | 1 | | | | | 1 | |
| Polymethacrylates | | | | 1 | | | | | 1 | |
| Polyoxyl 23 lauryl ether, | | 1 | | | | | | | | |
| 20 cetostearyl ether, 10 | | | | | | | | | | |
| oleyl ether | | | | | | | | | | |
| Polyoxyl 40 stearate, 50 | | 1 | | | | | | | | |
| stearate | | | | | | | | | | |
| Polysorbate 20, 60, 80, 40 | | 1 | | | | | | | | |
| Polyvinyl acetate | | | | 1 | | | | | | |
| Copolymers | | | | | | | | | | |
| Polyvinyl alcohol | | | | 1 | | | | | 1 | |
| Polyvinyl pyrrolidone | | | | 1 | | | | | 1 | |
| Riboflavin | | | | | | | 1 | | | |
| Castor oil | | | | | | | | 1 | | |
| Saccharose | | | | 1 | | 1 | | | | |
| Sorbitan monooleate, | | 1 | | | | | | | | |
| palmitate, stearate, | | | | | | | | | | |
| trioleate, tristearate, | | | | | | | | | | |
| laurate | | | | | | | | | | |
| Sorbitol | | | | | 1 | 1 | | 1 | | |
| Starch | | | | 1 | | | | | 1 | |
| Starches, rice, corn, | | | | | | | | | | |
| potato, wheat | | | | | | | | | | |
| Stearic acid | | 1 | | | | | | | | |
| Stearyl alcohol | | 1 | | | | | | | | |
| Talcum | | | | | | | | | 1 | |
| Titanium dioxide | | | | | | | 1 | | | |
| Tragcanth | | | | 1 | | | | | | |
| Triacetin | 1 | | | | | | | 1 | | |
| Triethanolamine | | | 1 | | | | | | | |
| Triethyl citrate | | | | | | | | 1 | | |
| Trometamol | | | 1 | | | | | | | |
| Vaseline | | | | | | | | | 1 | |
| Tartaric acid | | | 1 | | | | | | | |
| Urea | | | 1 | 1 | 1 | | | | | |
| Ceramide | | 1 | | | | | | | | |
| Hyaluronic acid | | | | 1 | | | | | | |

According to a further aspect of the use according to the invention, in particular of the at least one effective Pyridine-3,4-diamine, derivatives and/or at least one physiologically tolerable salt or the composition according to the invention, a dose of greater than or equal to 1 mg, greater than or equal to 2 mg, greater than or equal to 3, preferably greater than or equal to 5 mg to less than or equal to 60 mg per day, particularly preferably of 5 mg to 20 mg, preferably of 5 mg to 15 mg, in particular per 24 hours, is administered.

The approved maximum daily dose of the Pyridine-3,4-diamine, which is approved as drug, derivatives and/or at least one physiologically tolerable salt is 60 mg and the single dose may be 20 mg. A preferred maximum daily dose of the active ingredient Pyridine-3,4-diamine is 20 mg and/or a single dose of 5 mg to 10 mg, a maximum daily dose of the active ingredient Pyridine-3,4-diamine of 15 mg and/or a single dose 5 mg to 10 mg, preferably of 5 mg, is particularly preferred.

A further aspect of the use according to the invention is thus that a maximum daily dose has less than or equal to 60 mg, preferably of less than or equal to 40 mg, less than or equal to 30 mg, less than or equal to 20 mg, particularly preferably less than or equal to 15 mg, is administered in particular, preferably greater than or equal to one to less than or equal to five doses (single dosages) per day are administered, one dose particularly preferably contains greater than or equal to 2 mg, greater than or equal to 2.5 mg greater than or equal to 3 mg, greater than or equal to 2 mg to 7 mg, preferably greater than or equal to 2 mg to 5 mg, more preferably greater than or equal to 5 mg to less than or equal to 20 mg, to less than or equal to 18 mg, preferably to less than or equal to 15 mg, in each case of the at least one active ingredient Pyridine-3,4-diamine, derivative and/or of the active ingredient as physiologically tolerable salt, hydrate, solvate, co-crystal and/or mixtures of at least two of the compounds.

In a further aspect of the use according to the invention, the treatment is a long-term treatment with an effective dose, the effective does is preferably adapted to the physiology of the patient and is geared towards reaching a Bell score of greater than or equal to 60, greater than or equal to 70, greater than or equal to 80, greater than or equal to 90 up to 100 and/or a strength score of at least 4. A strength score of 5 is considered to be normal, thus healthy, in the case of a strength score of 3, this is referred to as a patient needing care, who is bed-ridden (cannot scratch his nose). In the case of a strength score of 4, the strength is diminished/reduced.

A one-time dosage of 5 mg, in particular within 1 to 3 hours before going to sleep or going to bed, respectively, preferably within 0 to 60 minutes, is also a subject matter of the invention. In a preferred design, a subject matter of the invention is a one-time dosage of at least 5 mg to 25 mg, in particular within 1 to 3 hours, preferably within 0 to 60 minutes before going to sleep. The effect of the Amifampridine preferably lasts approx. 4 hours. The Bell score value preferably increases to at least 90 to 100 and/or the need for sleep decreases to below 11 hours, preferably to below 8 hours. The individual need for sleep also depends on whether Amifampridine (Pyridin-3,4-Diamine) to treat a fatigue, which is associated with a viral infection and optionally a neuroimmunological disease and/or a chronic fatigue syndrome appears in combination. Amifampridine can particularly preferably be provided as pharmaceutical composition for the use according to the invention as prolonged-release composition. For this purpose, Amifampridine can be present as co-crystal in the formulation for the prolonged release and/or a prolonged-release composition can be provided, in the case of which the formulation of the excipients and/or of the capsule effect a prolonged release.

After withdrawal trials (three days no Amifampridine) with recurring fatigue long-term medication with a daily dose of 20 mg to 30 mg, preferably of 20 to 25 mg of 5 mg each at an interval of 4 hours, whereby the Bell score is from 90 to 100 in the long term. This Bell score value can already be held for three quarters of a year. Taken from morning to night before going to sleep.

The above-mentioned, improved Bell score is preferably reached within less than 3 days, less than 2 days. Preferably within 24 hours and particularly preferably within a few hours. Due to the fact that the at least one Pyridine-3,4-diamine, derivatives and/or at least one physiologically tolerable salt, hydrate, solvate, co-crystal and/or mixtures thereof, preferably in the composition according to the invention, takes effect quickly, within a few hours (1, 2, 3, 4, 5 to 8) or immediately, preferably almost immediately, the absence of an effect suggests that a dose titration is required in order to adapt the appropriate and effective dose to the physiology of the patient (weight, size and/or metabolic activity). In the case of an appropriate dose for the respective patient, it should take effect immediately.

In a further aspect of the use according to the invention, the long-term treatment lasts greater than or equal to two weeks and can transition into a permanent treatment. In a further aspect of the use according to the invention, the treatment achieves a Bell score of greater than or equal to 60, greater than or equal to 70, greater than or equal to 80, or more, and the long-term treatment serves the purpose of maintaining a predefined Bell score. The predefined Bell score corresponds to the individual reference value of the patient prior to the viral infection. In the alternative, the predefined Bell score corresponds to the classification, which the patient perceives to be sufficient or to be appropriate and which is made with the treating physician. This results in a Bell score, which follows the described features. During the course of the treatment, acute treatment, long-term treatment and long-term treatment, it can be desirable to adapt, to increase or to lower the dose, in order to achieve and maintain the desired Bell score.

A method for the treatment of a fatigue, which is associated with a viral infection according to any one of claims 1 to 13 is also a subject matter of the invention.

The Bell-score (or scale) is a measure of the impediment in the case of ME & CFS. It is also suitable for long COVID patients. The Bell scale is individual because each patient has a different stress tolerance and suffering from severe symptoms at rest can be relatively different.
100 No symptoms at rest; no symptoms with physical stress; a normal overall activity level; able to work full-time without any difficulties.
90 No symptoms at rest; mild symptoms with physical and mental stress; a normal overall activity level; able to work full-time without any difficulties.
80 Mild symptoms at rest; the symptoms intensify due to stress; a slight performance limitation noticeable only with activities, which are strenuous; able to work full-time in jobs, which require exertion of force, with difficulties.
70 Mild symptoms at rest; significant limitations noticeable in the daily activities; the functional state is a total of about 90% of the norm—with the exception of activities, which require an exertion of force; able to work full-time with difficulties.
60 Mild symptoms at rest; significant limitations in the daily activities; the functional state is a total of about 70%-90% of the norm; unable to work a full-time job if physical work is required there; however, able to work full-time if it is light work and the hours of work can be handled flexibly.
50 Moderate symptoms at rest; moderate to severe symptoms in response to physical stress or activity; the functional state is reduced to 70% of the norm; unable to perform strenuous work, but able to perform light work or desk work for 4-5 hours daily, whereby rest periods are required.
40 Moderate symptoms at rest; moderate to severe symptoms in response to stress or activity; the functional state is reduced to 50%-70% of the norm; unable to perform strenuous work, but able to perform light work or desk work for 3-4 hours daily, whereby rest periods are required.
30 Moderate to severe symptoms at rest; severe symptoms in response to any stress or activity; the functional state is reduced to 50% of the norm; generally housebound; unable to perform strenuous work, but able to perform light work or desk work for 2-3 hours daily, whereby rest periods are required.
20 Moderate to severe symptoms at rest; severe symptoms in response to any stress or activity; the functional state is reduced to 30%-50% of the norm, except for rare exceptions, unable to leave the house; bedridden for the majority of the day; unable to concentrate for more than one hour per day.
10 Severe symptoms at rest; bedridden most of the time; leaving the house is not possible; significant cognitive symptoms, which make it impossible to concentrate.
0 Constant severe symptoms; always bedridden; unable to perform the simplest care measures.

The use according to the invention of Aminopyridine, preferably in a corresponding composition, results an immediate improvement of the muscular deficits and further symptomatology, as demonstrated by the following examples.

### EXAMPLES

Pyridine-3,4-diamine Amifampridine (formulation as finished drug) was administered in the below-mentioned application examples. As part of the double-blind study, capsules that look the same with Amifampridine or placebo were prepared blind by a pharmacy, whereby only the pharmacy knew which capsules contained active ingredient or placebo.

In the case of the patients described in case 1, Aminopyridine, here 3,4-Daminopyridine, did not only effect an immediate improvement of the muscular deficits, but also a sudden improvement of a serious fatigue symptomatology that existed since the COVID infection, with a need for sleep or rest, respectively, of up to 20 hours per day (brain fog) as well as pronounced lack of concentration.

In case 2, a patient is described, who likewise suffered from a serious fatigue symptomatology after COVID infection 5/22, an assignable neurological or internal underlying disease did not exist. The administration of 5 mg of Amifampridine followed, a complete improvement of the fatigue symptomatology as well as complete restitution of the ability to concentrate could be seen thereby. Slight perioral paresthesias occurred as assignable side effects, which were not perceived as being bothersome. The following laboratory and ECG controls for detecting substance-associated complications were inconspicuous. The off-label therapy was continued with a daily dose of 3×5 mg, the existing fatigue ceases completely thereby, complete performance exists again.

In case 3, a patient is described, who suffered from fatigue symptomatology with a need for sleep of up to 2×9 hours per day, after being infected with COVID twice. An off-label therapy trial with 5 mg of Amifampridine also took place here, which led to a sudden improvement of the ability to concentrate as well as physiological need for sleep and rest, in the same way as in the above-described cases. Transient perioral paresthesias with very low intensity were also noticed here.

According to the previous clinical observations, the Amifampridine obviously has a good efficacy in the case of fatigue symptomatology without the presence of a neurological underlying disease, which has not been described in this form until now.

It is known that in the case of LEMS patients, Amifampridine (Diaminpyridine) leads to an improvement of a co-indicated fatigue of LEMS patients, whereby studies to that effect are not available due to the low incidence of LEMS patients (1:100.000). For the chemically closely-related Fampridine (Monoaminpyridine), which is used in MS patients, the improvement of an accompanying fatigue symptomatology is described in the literature. It is currently not sufficiently clarified whether the described positive influences on a fatigue symptomatology in the case of Aminopyridine is a substance-specific effect as part of the neurological underlying disease. According to the described cases, an unspecific positive effect on the fatigue is to be assumed, which has to still be clarified by means of further research, but an efficacy in the case of fatigue symptomatology as part of a long COVID syndrome can be derived from the case descriptions presented here.

The Lambert-Eaton myasthenic syndrome (LEMS) is a rare disease. It often appears as part of a tumor disease (SCLC) or more rarely as part of autoimmune diseases. LEMS with small cell lung cancer is referred to as SCLC-LEMS and the nontumor LEMS as NT-LEMS. In the case of 50 to 60% of patients, SCLC-LEMS is detected together with a neuroendocrine tumor associated with smoking. A few reports also describe LEMS in connection with other tumors. These tumors then often have a neuroendocrine character. Examples for these other lung tumors or prostate cancer, thus the most common extrathoracic neuroendocrine tumor, which is commonly associated with an anti-Hu syndrome.

LEMS is a disruption of the impulse transmission between nerve and muscle and thus leads to a weakness of the musculature. The muscle weakness in the case of LEMS is triggered by a misdirected immune reaction. This is so because antibodies against endogenous, voltage-gated calcium channels of the P/Q type are formed, which are located at the impulse transmission point (synapsis). The antibody formation reduces the release of the neurotransmitter Acetylcholine at the synapses of the motoric and autonomous nervous system and thus disrupts the transmission line.

LEMS is thus a presynaptic, neuro-muscular impulse transmission disruption. As described above, the Lambert-Eaton myasthenic syndrome is often associated with a tumor disease (paraneoplastic LEMS). LEMS can thereby precede the tumor diagnosis by two to maximally five years and can contribute to the early detection of the tumor. The most common cancerous disease in the lung is the small bronchogenic carcinoma thereby, for which smoking has been proven to be the trigger. However, other tumors of the lung, of the prostate, of the thymus or of other organs can also be associated with an LEMS. LEMS can likewise appear as an autonomous autoimmune disease independently of tumor diseases. LEMS is a rare disease with 1 sick person to 250,000 to 300,000 people.

Different concentrations, daily doses as well as number of the doses are analyzed in further studies in relation to the Bell score before and after the treatment for a defined period of time.

Complementary parameters can be used for this purpose, which are in fact suitable to qualify and to quantify the symptoms, such as sleep disorders, tiredness, increased need for sleep, muscular weakness, decreased physical endurance, decreased mental endurance, decreased recollection ability, impairment in the ability to think and remember, loss of concentration, increased pain perception and/or an attention deficit. For this, biochemical parameters can be combined, which are suitable to analyze circulation, hormone and/or immune system, comprise the measurement of the hormones, e.g., melatonin, cortisol and/or serotonin, the measurement of the upper, systolic and of the lower, diastolic value, determination of the blood sugar, etc.

A further study was performed on a patient group. A reduced fatigue symptomatology was determined in this study as well in the case of post-COVID syndrome under Amifampridine.

The prevalence of a post-COVID syndrome (PCS) is 10-15% after SARS-CoV-2 infection, a large variety of functional symptoms appear in addition to specific organ diseases, predominantly a fatigue symptomatology. The latter is characterized by persistent tiredness, weakness, cognitive deficits as well as increased need for sleep. The effect of Amifampridine in patients with PCS, who suffered from unphysiologically increased need for sleep as well as limited cognition, is described in this study or case series, respectively. The Bell score, which focusses on the limitation of the vigilance, physical endurance and job, was used for the evaluation.
In the case of pat. 1 of the further study, a previous Zn. EBV infection is known, otherwise no internal or neurological preexisting conditions. Fluctuating fatigue symptomatology after SARS-CoV-2 infection in May of 2022 with a Bell score between 20 and 40, increased need for sleep with 12 hours/24 h. After taking 5 mg of Amifampridine three times within 24 h (h: hours), there was complete convalescence with a Bell score of 100, the need for sleep reduced to 7 hours/24 h. Withdrawal trials ended in a fatigue again.
In the case of pat. 2, of the further study, a 60-year-old patient, a severe fatigue symptomatology with severe limitation of the professional activity appeared after being infected with SARs-Cov-2 twice. Bell score 40, the need for sleep was 12 hours/24 h, limited vigilance in the waking phases. After administering 5 mg of Amifampridine three times, a Bell score of 100 was reached, the need for sleep decreased to 7.5 hours/24 h, unlimited vigilance. The same effects in the case of recurrent fatigue.
In the case of pat. 3, of the further study, a 26-year-old patient, limited ability to concentrate, need for sleep 11 to 12 hours/24 h, Bell score 40 existed after being infected with SARS-COV-2 2022 twice. After spontaneously taking 5 mg of Amifampridine, a reduction of the need for sleep to 7-9 hours occurred, Bell score 100. The effect lasted about 36 hours and was reproducible by taking Amifampridine again.
Pat. 4, of the further study, a 36-year-old patient, who took care of severely ill people in an intensive care unit during the first COVID wave, shortly thereafter typical symptomatology with loss of taste and post-COVID myocarditis. Pronounced limitation of the physical endurance (Bell score 20) as well as increased need for sleep to 12 to 14 hours (h)/24 hours (h). After taking 5 mg of Amifampridine three times each within 24 h as daily dose, fatigue symptomatology is declining and muscular symptoms (Bell score 50) are improving and the need for speed is decreasing to 8 to 10 hours/24 h. The effect was reproducible. The limitation of the endurance, which still exists, is due to the cardiac issue.
In the case of the 63-year-old pat. 5, of the further study, a neuromuscular disease (Lambert-Eaton myasthenic syndrome synonymously to the Lambert-Eaton myasthenic syndrome) existed since 2021, severe fatigue, need for sleep 16 to 18 hours/24 h, Bell score 10 after SARS-CoV-2 infection 5/22. After administering 5 mg of Amifampridine three or four times as daily dose, the fatigue ceased, need for sleep 8-9 hours/24 h, improved muscular endurance.

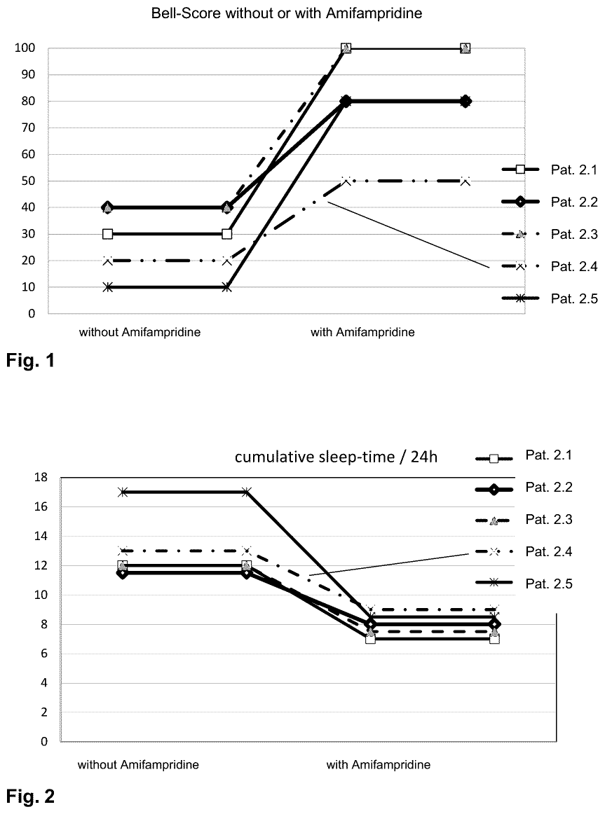

The following dosages of patients 2.1 to 2.5 and the changed Bell score values as well as the need for sleep in hours are illustrated in FIGS. 1 and 2 (FIG. 1 Bell-score with and without Amifampridine, patients 2.1 to 2.5, FIG. 2: cumulative sleep time in 24 h).

In the case of pat. 2.1, a 61-year-old psychotherapist without internal or neurological preexisting conditions, fluctuating fatigue symptomatology with a Bell score between 20 and 40 as well as increased need for sleep with 12 hours/24 h occurred after SARS-CoV-2 infection in May of 2022. After taking 5 mg of Amifampridine once, there was complete convalescence with a Bell score of 100, the need for sleep reduced to 7 hours/24 h.

After withdrawal trials (three days no Amifampridine) with recurrent fatigue, a long-term medication with a daily dose of 20 mg to 25 mg of 5 mg each at an interval of 4 hours occurred, whereby the Bell score is permanently from 90 to 100. This Bell score value can already be maintained for three quarters of a year. Taken from morning to night before going to sleep.

In the case of pat. 2.2, a 26-year-old medical student, there was limited ability to concentrate, need for sleep 11-12 hours/24 h, Bell score 40 after being infected with SARS-CoV-2 2022 twice. After taking 5 mg of Amifampridine, reduction of the need for sleep to 7-9 hours, Bell score 80. The effect lasted about 36 hours and was reproducible by taking Amifampridine again, subsequent long-term medication with a daily dose of 20 mg to 25 mg of 5 mg each at an interval of 4 hours, whereby the Bell score is permanently from 90 to 100. This value can already be reproduced for the past month.

In the case of pat. 2.3, a 60-year-old attorney, a severe fatigue symptomatology occurred after being infected with SARS-CoV-2 twice. Bell score 40, the need for sleep was 12 hours/24 h, limited vigilance in the waking phases. After 5 mg of Amifampridine, Bell score 100, need for sleep 7.5 hours/24 h, unlimited vigilance. Identical effects in the case of recurrent fatigue, when further taking 5 mg of Amifampridine as needed.

The 36-year-old patient 2.4 took care of severely ill people in an intensive care unit during the first COVID wave, typical symptomatology shortly thereafter with loss of taste, muscular symptoms and post-COVID myocarditis. Pronounced limitation of the physical endurance (Bell score 20) as well as increased need for sleep to 12-14 hours/24 h. After 5 mg of Amifampridine, declining fatigue symptomology and muscular symptoms (Bell score 50) as well as decrease of the need for sleep to 8 to 10 hours/24 h. The effect was reproducible. If needed, this patient individually takes 5 mg of Amifampridine each at an interval of weeks, in order to improve the Bell score 60 and the fatigue symptomology.

The still existing limitation of the endurance is due to the cardiac issue.

Pat. 2.5:63-year-old cardiologist, suffered from a neuromuscular disease (Lambert-Eaton myasthenic syndrome) since 2021, after SARS-CoV-2 infection 5/22 severe fatigue, need for sleep 16-18 hours/24 h, Bell score 10. After an initial administration of 3×5 mg of Amifampridine, the fatigue ceased, need for sleep 8-9 hours/24 h, improved muscular endurance, Bell score 80. In spite of progressing LEMS, still improved fatigue. For a period of about 9 months, up to 60 mg and for the last two weeks 70 mg of Amifampridine were taken with the underlying disease LEMS. At the beginning, an improvement of the Bell score could already be reached with 15 mg per day. In the course of the above-mentioned months, the dose was increased to 60 mg and later to 70 mg per day. An improvement was initially achieved with 3 times of a 5 mg daily dose. Subsequently, the individual dose was 3 times 20 mg each and optionally additionally an individual dose of 5 mg. The intervals between the 20 mg doses between getting up and going to bed are approximately evenly distributed at approx. 6 to 7 hours and, if needed, additionally further 5 mg of Amifampridine. The fatigue symptomology is thus controlled satisfactorily, Bell score 70 to 80 with regard to fatigue and with regard to the muscular deficits a Bell score of approx. 20. Prior to the administration of the Amifampridine, the Bell score was 20 to 30 with regard to the fatigue and overall condition.

In the case of pat. 2.1 and 2.2, a long-term medication with 20 mg of Amifampridine each per day were required for reconvalescence and thus offered prerequisites for a double-blind withdrawal trial for 3 days each.

Pat. 2.1 with verum on days 1-3, followed by placebo on days 4-6:

Pat. (pat. 2.1) with Bell score 90 already started with Amifampridine, as described above, and continued this with blister with 6 daily doses each day, one drawer with capsules for 3 days with 4 times 5 mg of Amifampridine each. The dosage for 3 days each was placebo with 4 times placebo capsules each for patient 1 and 2 (verum and placebo).

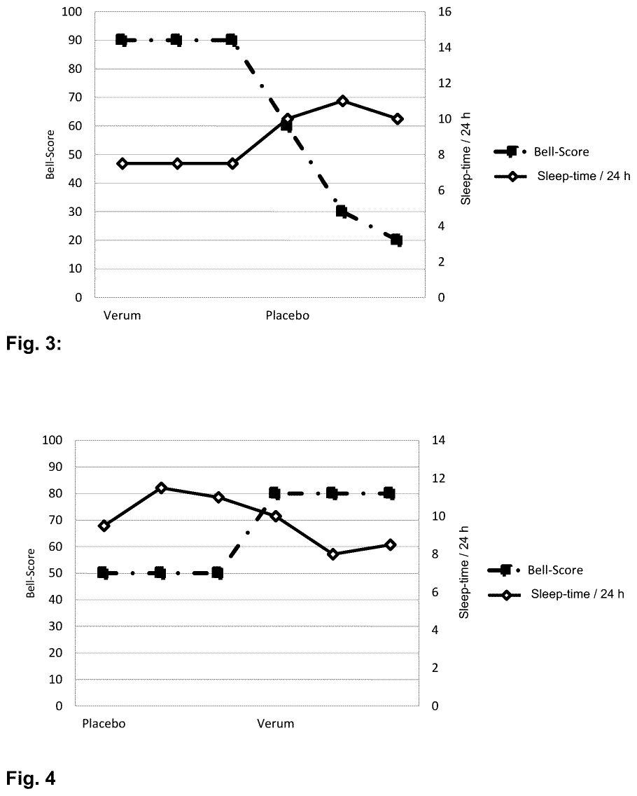

Pat. 2.1: verum on days 1-3, followed by placebo on days 4-6: reduction of the Bell score to minimally 30 in the placebo phase, increased sleep time to 10.5 hours (FIG. 3: pat. 2.1, Bell score (line with squares with side lines of the squares parallel and perpendicular to the main line; sleep time/24 h (line with smaller squares, tips of squares point upwards/downwards as well as to the right and left).

Pat. 2.2: placebo on days 1-3, verum on days 4-6: reduction of the Bell score under placebo to 50, increased sleep time to 11 hours (FIG. 4: pat. 2.2, Bell score (line with square, side line parallel to the main line, sleep time/24 h (line with smaller square, tips point upwards/downwards as well as to the right and left).

Further double-blind studies are planned with patients with an initial dose: of 3×5 mg of Amifampridine and if not effective with a dose of 3×10 mg of Amifampridine for a duration of two weeks. The control group receives a placebo.

Fatigue is concomitant phenomenon in the case of internal, neurological and oncological diseases with significant loss of the quality of life. In the case of increasing incidence of PCS, frequent inability to work and disability exist as a result of fatigue.

The molecular mechanisms are not yet decoded, favorable effects of the Fampridine (Monoaminopyridine) on fatigue in the therapy of multiple sclerosis are known with regard to symptomatic therapy approaches. On the part of the University Hospital of Basel, a study for this with regard to the question of the influence of Fampridine on the working memory is performed. Amifampridine is indicated as orphan drug singularly for the Lambert-Eaton myasthenic syndrome as in the case of pat. 5. Improvement of a coincident fatigue as phosphates with LEMS are described for 20 to 80 mg as daily doses (Clinical Trial J Clin Neuromuscul Dis. 2019 March; 20 (3): 111-119: Shieh, Perry; Sharma, Khema, Kohrman, Bruce; Oh, Shin J: Amifampridine Phosphate (Firdapse) Is Effective in a Confirmatory Phase 3 Clinical Trial in LEMS). In all described cases according to the invention, there was a significant reduction of the fatigue triggered by a viral infection by ss(+) RNA viruses of the family Coronavirdidae as well as of the unphysiologically increased need for sleep triggered by these viruses, a low-dosed administration of, in particular one-time to four-time administration of 5 mg as daily dose, preferably of a one-time to three-time daily dose, the effects were reproducible in withdrawal trials. Further controlled studies are required.

A use of Pyridine-3,4-diamine, one of its derivatives and/or of a physiologically tolerable salt thereof for the prevention and/or treatment of a fatigue, which is associated with a viral infection or analogously product(s) for the above- and below-mentioned use, is also a subject matter of the invention.

In each case independently an above use, in which the viral infection is triggered by ss(+) RNA viruses of the family Coronaviridae, preferably by the virus SARS-CoV-1, SARS-CoV-2, MERS-COV, HCoV-HKU1, HCoV-OC43, HCoV-NL63 and/or HCoV-229E, or the family of the Herpesviridae comprising Epstein-Barr virus.

In each case independently an above use, in which the viral infection is a SARS-CoV-2 infection.

In each case independently an above use, in which the fatigue is a post-infectious fatigue, preferably after abatement of the viral infection occurs, preferably after determining a Ct value of greater than or equal to 30 and/or a complex neuroimmunological disease comprising myalgic encephalomyelitis and/or chronic fatigue syndrome (ME/CFS), preferably of a post-infectious fatigue syndrome.

In each case independently an above use, in which the health symptoms of the acute disease phase of a viral, preferably SARS-CoV-2, infection, continue for greater than or equal to 3, greater than or equal to 4 weeks, or appear again.

In each case independently an above use, in which the symptoms of the acute disease phase of a viral, preferably SARS-CoV-2, infection continue for more than 4, preferably more than 5, more than 6, more than 7, more than 8, more than 9, more than 10, more than 11 or more than 12 weeks after onset of the viral, preferably SARS-CoV-2, infection.

In each case independently an above use, in which the fatigue comprises symptoms selected from sleep disorders, tiredness, increased need for sleep, muscular weakness, decreased physical endurance, decreased mental endurance, decreased recollection ability, impairment in the ability to think and remember, loss of concentration, increased pain perception and/or an attention deficit and/or dysregulations of circulation, hormone and/or of the immune system. In each case independently an above use, in which the fatigue is present with a Bell score of less than or equal to 50, less than or equal to 40, less than or equal to 30, less than or equal to 20.

In each case independently an above use, in which the need for sleep is reduced to physiological hours of greater than or equal to 6 to less than or equal to 12, preferably greater than or equal to 6, greater than or equal to 7, greater than or equal to 8 to less than or equal to 11 hours, less than or equal to 10, less than or equal to 9, particularly preferably greater than or equal to 6 to less than 8.

A composition containing at least one Pyridine-3,4-diamine, at least one of its derivatives and/or at least one physiologically tolerable salt for the use in the prevention and/or treatment of a fatigue, in each case independently for an above use, which is associated with a viral infection.

In each case independently an above use, in which a dose of greater than or equal to 1 mg, greater than or equal to 2 mg, greater than or equal to 3 mg, preferably greater than or equal to 5 mg to less than or equal to 60 mg per day, in particular of 1 mg to 30 mg, preferably of 2 mg to 20 mg per day, is administered.

In each case independently an above use, in which a maximum daily dose has less than or equal to 60 mg, preferably greater than or equal to one to less than or equal to five doses per day are administered, particularly preferably, a dose contains greater than or equal to 2, greater than or equal to 2.5 greater than or equal to 3 mg, greater than or equal to 5 mg to less than or equal to 20 mg, to less than or equal to 18 mg, preferably to less than or equal to 15 mg, in each case of the at least one effective Pyridine-3,4-diamine, derivative and/or of the physiologically tolerable salt.

In each case independently an above use, whereby the treatment is a long-term treatment with an effective dose, the effective dose is preferably adapted to the physiology of the patient and is geared towards reaching a Bell score of greater than or equal to 60, greater than or equal to 70, greater than or equal to 80, greater than or equal to 90 up to 100 and/or a strength level of at least 4.

In each case independently an above use of the product, whereby the long-term treatment lasts greater than or equal to two weeks and can transition into a long-term treatment. In each case independently an above use, whereby the treatment achieves a Bell score of greater than or equal to 60, greater than or equal to 70 greater than or equal to 80 or more and the long-term treatment serves the purpose of maintaining the Bell score.

Pyridine-3,4-diamine, a physiologically tolerable salt, solvate, hydrate, co-crystal, polymorphs thereof and/or mixtures containing two of the mentioned compounds for the use for the prophylaxis and/or treatment of a fatigue as described herein, which is associated with a viral infection and optionally i) for the prophylaxis and/or optionally ii) treatment of a complex neuroimmunological disease comprising myalgic encephalomyelitis and/or chronic fatigue syndrome (ME/CFS), preferably of a post-infectious fatigue syndrome, is particularly preferred.

Pyridine-3,4-diamine, a physiologically tolerable salt, solvate, hydrates, co-crystal, polymorphs thereof and/or mixtures containing two of the mentioned compounds for the respective above independent use is particularly preferred.

A pharmaceutical composition comprising Pyridine-3,4-diamine, a physiologically tolerable salt, solvate, hydrate, co-crystal, polymorphs thereof and/or mixtures containing two of the mentioned compounds as well as at least one pharmaceutical excipient for the use for the prophylaxis and/or treatment of a fatigue, as described herein, which is associated with a viral infection and/or for the prophylaxis and/or treatment of a complex neuroimmunological disease comprising myalgic encephalomyelitis and/or chronic fatigue syndrome (ME/CFS), preferably of a post-infectious fatigue syndrome, is likewise a subject matter of the invention. And in particular for the above respective independent use. A pharmaceutical composition for the respective independent use as described above, in particular for the prophylaxis and/or treatment of a fatigue, which is triggered with a viral infection by ss(+) RNA viruses of the family Coronaviridae, is further a subject matter of the invention.

Methods for the treatment or methodology for the treatment as described above and below are likewise a subject matter of the invention.

1. A method for the prophylaxis and/or treatment in a patient in need thereof of a fatigue, which is associated with a viral infection and optionally i) for the prophylaxis and/or treatment of a complex neuroimmunological disease comprising myalgic encephalomyelitis and/or optionally ii) of a chronic fatigue syndrome (ME/CFS), optionally of a post-infectious fatigue syndrome, said method comprising administering to said patient an effective amount therefor of a compound selected from the group consisting of pyridine-3,4-diamine, a physiologically tolerable salt, solvate, hydrate, co-crystal, or polymorph thereof, and mixtures containing two of the mentioned compounds.

2. Method according to claim 1, wherein the viral infection is triggered by ss(+) RNA viruses of the family Coronaviridae, optionally by the virus SARS-CoV-1, SARS-CoV-2, MERS-COV, HCoV-HKU1, HCoV-OC43, HCoV-NL63 and/or HCoV-229E, or the family of the Herpesviridae comprising Epstein-Barr virus.

3. Method according to claim 1, wherein the viral infection is a SARS-CoV-2 infection.

4. Method according to claim 1, wherein the fatigue is a post-infectious fatigue, optionally appears after abatement of the viral infection, optionally after determination of a Ct value of greater than or equal to 30 and optionally i) of a complex neuroimmunological disease comprising myalgic encephalomyelitis and/or optionally ii) chronic fatigue syndrome (ME/CFS), optionally of a post-infectious fatigue syndrome.

5. Method according to claim 1, wherein the health problems of the acute disease phase of a viral, optionally SARS-CoV-2, infection, persist for greater than or equal to 3, greater than or equal to 4 weeks, or appear again.

6. Method according to claim 1, wherein the fatigue comprises symptoms selected from sleep disorders, tiredness, increased need for sleep, muscular weakness, decreased physical endurance, decreased mental endurance, decreased recollection ability, impairment in the ability to think and remember, loss of concentration, increased pain perception and/or an attention deficit and/or dysregulations of the circulation, hormone and/or of the immune system.

7. Method according to claim 1, wherein the fatigue is present with a Bell score of less than or equal to 50, less than or equal to 40, less than or equal to 30, or less than or equal to 20.

8. Method according to claim 1, wherein the need for sleep is reduced to physiological hours of greater than or equal to 6 to less than or equal to 12, optionally greater than or equal to 6, greater than or equal to 7, greater than or equal to 8 to less than or equal to 11 hours, less than or equal to 10, less than or equal to 9, optionally greater than or equal to 6 to less than 8.

9. Method according to claim 1, wherein a dose of greater than or equal to 1 mg, greater than or equal to 2 mg, greater than or equal to 3 mg, optionally greater than or equal to 5 mg to less than or equal to 60 mg per day is administered.

10. Method according to claim 1, wherein a maximum daily dose has less than or equal to 60 mg, optionally greater than or equal to one to less than or equal to five doses per day are administered, a dose optionally contains greater than or equal to 2, greater than or equal to 2.5, greater than or equal to 3 mg, greater than or equal to 5 mg to less than or equal to 20 mg, to less than or equal to 18 mg, optionally to less than or equal to 15 mg, of the at least one effective Pyridine-3,4-diamine each, derivative and/or of the physiologically tolerable salt.

11. Method according to claim 1, wherein the treatment is a long-term treatment with an effective dose, the effective dose is optionally adapted to the physiology of the patient and is geared towards reaching a Bell score of greater than or equal to 60, greater than or equal to 70, greater than or equal to 80, greater than or equal to 90 up to 100 and/or a strength score of at least 4.

12. Method according to claim 1, wherein the long-term treatment lasts greater than or equal to two weeks and optionally transitions into a permanent treatment.

13. Method according to claim 1, wherein the treatment achieves a Bell score of greater than or equal to 60, greater than or equal to 70, greater than or equal to 80 or more and the long-term treatment serves the purpose of maintaining the Bell score.

14. A pharmaceutical composition comprising (a) an amount of a compound effective for the prophylaxis and/or treatment of a fatigue, which is associated with a viral infection and/or for the prophylaxis and/or treatment of a complex neuroimmunological disease comprising myalgic encephalomyelitis and/or chronic fatigue syndrome (ME/CFS), optionally of a post-infectious fatigue syndrome, wherein the compound is selected from the group consisting of pyridine-3,4-diamine, a physiologically tolerable salt, solvate, hydrate, co-crystal, or polymorph thereof, and mixtures containing two of the mentioned compounds as well as (b) at least one pharmaceutical excipient.

15. A method for the prophylaxis and/or treatment in a patient in need thereof of a fatigue, which is associated with a viral infection and optionally i) for the prophylaxis and/or treatment of a complex neuroimmunological disease comprising myalgic encephalomyelitis and/or optionally ii) of a chronic fatigue syndrome (ME/CFS), optionally of a post-infectious fatigue syndrome, said method comprising administering to said patient an effective amount therefor of the pharmaceutical composition according to claim 14.
